(19) United States
(12) Patent Application Publication
(10) Pub. No.: US 2018/0279985 A1
(43) Pub. Date: Oct. 4, 2018

(54) Non-Invasive Breast Cancer Detection Using Co-Registered Multimodal Probes: Microwave Nearfield Radar Imaging (NRI), Digital Breast Tomosynthesis (DBT), Ultrasound Imaging (US) And Thermoacoustic Imaging (TA)

(71) Applicant: Northeastern University, Boston, MA (US)

(72) Inventors: Jose A. Martinez-Lorenzo, Wellesley, MA (US); Richard Obermeier, East Boston, MA (US); Ashkan Ghanbarzadeh, Boston, MA (US); Ali Molaei, Boston, MA (US); Juan Heredia Juesas, Jamaica Plain, MA (US)

(21) Appl. No.: 15/765,699

(22) PCT Filed: Oct. 28, 2016
(86) PCT No.: PCT/US2016/059548

§ 371 Date: Apr. 3, 2018

Related U.S. Application Data
(60) Provisional application No. 62/413,873, filed on Oct. 27, 2016.
(60) Provisional application No. 62/412,671, filed on Oct. 25, 2016.
(60) Provisional application No. 62/248,041, filed on Oct. 29, 2015.

(51) Int. Cl. A61B 6/00; G06T 7/00; G06T 11/00; A61B 5/05; A61B 6/02; A61B 8/08; A61B 5/00

(52) U.S. Cl. CPC A61B 6/5247; G06T 7/0014; G06T 11/008; A61B 5/0507; A61B 6/025; A61B 6/502; G06T 2207/30068; A61B 8/0825; A61B 5/0093; A61B 5/7267; G06T 2207/10084; G06T 2207/20081; A61B 6/5217

(57) ABSTRACT

A cancer detection system may comprise at least two imaging systems, each of which implements a different imaging modality, and each of which provides sampled image data. The system may further include, for each imaging modality, a modeling unit to produce modeled image data based on a common set of biophysical parameters. The system may also include a joint non-linear inversion module to receive information from each modeling unit and reconstruct a set of joint biophysical properties. The system may include a scaling unit to revise the common set of biophysical parameters based on the set of joint biophysical properties. The system may include a comparator to compare the sampled image data from each of the imaging systems to the corresponding modeled image data to determine a difference between the sampled image data and the modeled image data, and to determine when the difference is less than a threshold difference.

### RELATED APPLICATION

This application claims the benefit of and priority to U.S. Provisional Application No. 62/413,873, filed on Oct. 27, 2016, U.S. Provisional Application No. 62/412,671, filed on Oct. 25, 2016 and U.S. Provisional Application No. 62/248,041, filed on Oct. 29, 2015. The entire teachings of the above applications are incorporated herein by reference.

### BACKGROUND

Conventional mammography screening is the only modality that has been shown to reduce the chance of death from breast cancer in randomized control trials. Depending on the breast tissue composition, however, the chances of detecting early breast cancer are different when using conventional screening mammograms.

The breast is made up of a mixture of tissues, including fibrous connective and glandular tissues, as well as fatty tissue. Radiologists classify breast density using a four level density scale, L1 through L4, where L1 describes the lowest breast density and L4 describes the highest breast density. Having breasts with relatively dense tissue (i.e., level 3 and 4) not only may increase the risk of getting breast cancer, but may also increase the difficulty of detecting breast cancer when using two-dimensional (2D), x-ray based screening mammograms. This is because the low contrast (conspicuity) between fibrous or glandular tissue and cancerous tissue is only of about 1 percent at radiological frequencies both generally appear white at x-ray frequencies. Women with breasts consisting of dense tissue may use adjunctive (additional) imaging test to effectively increase the conspicuity, in order to increase the likelihood of detecting early cancers.

One adjunctive imaging test is the bilateral whole breast sonography (i.e., ultrasound imaging). In women with dense breast (heterogeneous dense and extremely dense), screening ultrasound may detect additional cancers to those discovered with conventional mammography, similar to that seen with other adjunctive tests. Nevertheless, the number of unnecessary biopsies resulting from ultrasound imaging increases to an unacceptable level when it is used within a general population (i.e., dense and not dense breast) as a screening test. For this reason, ultrasound is only used as an adjunctive imaging test for women with an extremely dense breast.

Another adjunctive test is the three-dimensional (3D) mammography, also known as Digital Breast Thomosynthesis (DBT). Contrary to 2D Conventional Mammography (CM), DBT collects multiple views of the compressed breast, thus enabling a 3D reconstruction. DBT enhances the likelihood of finding tumors by removing overlapping breast tissues, i.e., those located at different 2D slices. The radiation dose to the breast resulting from the use of 2D mammography and DBT is increased by a factor of 2 as compared to CM alone, although the radiation dose is still below the U.S. Food and Drug Administration (FDA) limit.

Another adjunctive imaging modality is the bilateral breast magnetic resonance imaging (MRI), although MRI requires an intravenous injection of a contrasting agent and introduces an elevated cost.

Existing modalities used for early detection of breast cancer suffer from several limitations. First, existing modalities are not optimal to analyze all four levels of breast density (L1-L4). Second, they require the use of either ionizing radiation (e.g., X-ray based), which may have potential carcinogenic effects, or potentially hazardous contrasting agents (e.g., MRI-based). Third, current CM requires the use of adjunctive imaging modalities for dense breasts (L3-L4), and adjunctive imaging may result in additional cost, unnecessary anxiety and biopsies. Fourth, current imaging modalities and their associated data analytic tools do not take into consideration the particular specifics of the patients, such as positive genetic mutations (e.g., BRCA1 and BRCA2), breast density, family history, among others. Fifth, current modalities are not capable of providing low cost and size equipment that features high contrast and resolution. Sixth, in the case of multimodal systems that provide additional information concerning the breast tissue composition (such as density, compressibility, viscosity, dielectric constant, and conductivity) to 2D/3D mammography or MRI, such multimodal systems are not capable of collecting all data in a single and quick session, and in a co-registered fashion.

X-ray based technologies such as CM and DBT are most often used to detect cancerous lesions within the breast. Unfortunately, these systems both suffer from the aforementioned low radiological contrast between healthy breast tissue and cancerous tissue. As a result, these technologies tend to produce a large number of false positives when used for early detection.

Nearfield Radar Imaging (NRI) is a less common technology for breast cancer detection that uses non-ionizing microwave radiation to assess the breast tissue. NRI is an appealing technology for breast cancer detection because, at microwave frequencies, the contrast between healthy breast tissue and cancerous tissue is on the order of 10 percent. Unfortunately, standalone NRI systems typically struggle to accurately detect cancerous lesions due to the heterogeneous distribution of tissues within the breast, having a wide dynamic range in the relative dielectric constants that ranges from 5 (fatty) up to 55 (fibrous) for healthy tissues, and about 60 for cancerous tissues at around 1 GHz.

### SUMMARY OF THE INVENTION

The described embodiments relate to the use of a hybrid, multimode, breast cancer detection system. The detection system may implement a fusion of different imaging modalities, for example Digital Breast Tomosynthesis (DBT) together with Microwave Nearfield Radar Imaging (NRI). In such a hybrid system, the DBT reconstruction may be used in order to form a prior distribution of tissues in the breast that can be used as a starting point for the NRI inversion process, although other imaging modalities may be used to provide this starting point. A linear linearized matrix of the non-linear in nature sensing problem may be formulated and solved, by applying for example the Born Approximation, to provide the complex permittivity of the breast tissues, although other techniques like iterative Born or the Contrast Source method can be used in the process of inverting the non-linear problem.

The described embodiments may detect cancerous tissues, or morphologically atypical tissues in general, with respect to healthy tissue. Morphologically atypical tissue may include, but is not limited to, cancerous tissue, tumorous tissue, precancerous tissue, and invasive tissue.

In addition, the reconstruction process may be formulated as a sparse recovery problem, such that certain compressive sensing (CS) techniques can be applied. See, for example, PCT/US16/25274 (entitled “Compressive Coded Antenna/Meta-Antenna,” filed on Mar. 31, 2016, the contents of which are incorporated by reference in their entirety) for examples of such CS techniques.

Imaging results, using real DBT data and synthetic NRI data demonstrate a tremendous potential for this technique in terms of finding tumors surrounded in fibroglandular tissue.

In the described embodiments, a multimode hybrid cancer detection system may include a fusion of two or more of (i) NM, (ii) DBT, (iii) Ultrasound Imaging (USI) and (iv) Thermoacoustic Imaging (TAI). In such hybrid embodiments, the fusion of the two or more modes is accomplished by co-registering the various modes' scans, so that the scans of all modes are captured with respect to the same physical configuration of the breast under study. The fusion is further accomplished by evaluating the captured multimodal scan data jointly rather than independently. An example of a hybrid DBT and NM system may be seen in PCT/US2014/042842, which is incorporated herein by reference in its entirety.

The captured multimodal scan data is collected while the breast is under clinical compression. Then, this data is used to reconstruct the pixel-based biophysical parameters of the breast—which may be given, for example, in terms of the porosity, fluid saturation, and solid matrix composition by using forward models for each modality and solving a joint non-linear inverse problem. The inversion makes use of a biophysical model that relates the biophysical parameters and the constitutive parameters for the different technologies.

The constitutive parameters may include the following:
For NRI—permittivity, permeability and conductivity
For DPT—absorption
For USI—density, bulk modulus, attenuation factor
For TAI—volumetric expansion coefficient, specific heat capacity

The biophysical parameters (porosity, fluid saturation, and solid matrix composition) uniquely identify different types of tissues inside of the breast for each pixel in which it is discretized: fatty tissue, fibrous tissue, cancerous tissues, calcifications, and so on. The multi-modal system is more capable of finding tumors at earlier stages of the disease due to at least the following reasons:
1. The ill-posedness of the intrinsically non-linear inverse problem is reduced by using a-priori information, which is used to define a near-to-optimal first guess of the true biophysical parameters.
2. The sensitivity and specificity of the multimodal sensor is higher than any other one working in a standalone mode, for example the high resolution DBT and/or USI images that presents a low radiological contrast between fibro-glandular and cancerous tissue can be combined with the high contrast but low resolution NM image to produce a fused NM/DBT/USI image with high resolution and contrast.
3. The joint inversion in the (low-dimensional) space of the biophysical parameters enables a consensus-type of solution for all modalities, and it reduces the impact of the degrading effect associated with solving the inverse problem in the (high-dimensional) space of the constitutive parameters of each modality.
4. Using an NM/UST/TAT or the NRI/USI modalities avoids the use of ionizing radiation associated with X-ray based DBT, while keeping high resolution and high fibrous-to-cancerous contrast, and thus providing high sensitivity and specificity.

In one aspect, the invention is a cancer detection system, comprising at least two imaging systems, each of which implements an imaging modality different from others of the at least two imaging systems, and each of which provides sampled image data based on its modality. The cancer detection may further include a processor and a memory with computer code instructions stored thereon, the memory is operatively coupled to the processor such that the computer code instructions, when executed by the processor, may cause the system to implement:
(a) for each imaging modality, a modeling unit configured to produce modeled image data for that modality based on a common set of biophysical parameters, (b) a joint non-linear inversion module configured to receive information from each modeling unit and reconstruct a set of joint biophysical properties based on the information from the modeling units, and (c) a scaling unit configured to revise the common set of biophysical parameters based on the set of joint biophysical properties. The cancer detection system may further include a comparator configured to compare the sampled image data from each of the imaging systems to the corresponding modeled image data to determine a difference between the sampled image data and the modeled image data and to determine when the difference is less than a threshold difference, thereby indicating that the sampled image data and the modeled image data has converged. The cancer detection system may further include a classifier configured to classify tissues corresponding to the image data as healthy or cancerous, based on the set of joint biophysical properties corresponding to the modeled image data upon convergence.

In one embodiment, the at least two imaging systems may include a Digital Breast Tomosynthesis (DBT) system and a Microwave Nearfield Radar Imaging (NRI) system.

In another embodiment, the at least two imaging systems includes two or more of (i) a Digital Breast Tomosynthesis (DBT) system, (ii) a Microwave Nearfield Radar Imaging (NRI) system, (iii) a UltraSound Imaging (USI) system, and a (iv) Thermoacoustic Imaging (TAI) system.

In another embodiment, the information from each modeling unit may include biological tissue parameters. The biological tissue parameters may include one or more of (i) electrical permittivity, (ii) permeability (iii) conductivity, (iv) elastic bulk modulus, (v) density, (vi) attenuation, (vii) thermodynamic heat capacity (viii) volumetric expansion coefficient, and (ix) radiological X-ray absorption.

In one embodiment, each modeling unit may include a biophysical model, a constitutive model, a forward model, and a field simulating model.

In another embodiment, the classifier may further classify tissues corresponding to the image data based on an unmixed version of the set of joint biophysical properties.

In another embodiment, the classifier may utilize a machine learning procedure to classify the tissues corresponding to the image data. The classifier may utilize a Quadratic Discriminant Analysis procedure (or other supervised learning classifier know in the art) to classify the tissues corresponding to the image data.

In one embodiment, the at least two imaging systems may reside on a mechatronic system that is integrated with a Digital Breast Tomosynthesis (DBT) system, such that all captured image data is co-registered.

In another aspect, the invention is a method of detecting cancer, comprising performing an imaging modality that is different from others of the at least two imaging systems, using each of at least two imaging systems. The method further includes providing, from each of the at least two imaging systems, sampled image data that is based on the image system's modality. The method may further include using a processor and a memory with computer code instructions stored thereon, producing modeled image data, for each imaging modality, based on a common set of biophysical parameters, reconstructing, based on information received from each modeling unit, a set of joint biophysical properties, and revising the common set of biophysical parameters based on the set of joint biophysical properties. The method may further comprise comparing the sampled image data from each of the imaging systems to the corresponding modeled image data to determine a difference between the sampled image data and the modeled image data and determining when the difference is less than a threshold difference, and classifying tissues corresponding to the image data as healthy or cancerous, based on the set of joint biophysical properties.

One embodiment may further include sequentially activating each of the imaging systems while a test subject remains clinically advantageous position.

Another embodiment may further include mechanically rotating sensors of the two or more imaging systems, in conjunction with the activating, to accomplish co-registration of the two or more imaging systems.

Another embodiment may further include classifying tissues corresponding to the image data using a machine learning procedure.

One embodiment may further include classifying tissues corresponding to the image data using a Quadratic Discriminant Analysis procedure.

Another embodiment may further include implementing, for each imaging modality, a forward model that simulates fields corresponding to the imaging modality.

Another embodiment may further include repeatedly revising the common set of biophysical parameters until the difference between the sampled image data and the modeled image data is less than a threshold difference.

In one embodiment, the set of joint biophysical properties include one or more of (i) electrical permittivity, (ii) permeability (iii) conductivity, (iv) elastic bulk modulus, (v) density, (vi) attenuation, (vii) thermodynamic heat capacity (viii) volumetric expansion coefficient, and (ix) radiological X-ray absorption.

In another embodiment, processing modeled image data for each modality is accomplished with a biophysical model, a constitutive model, a forward model, and a field simulating model.

In another embodiment, performing the imaging modality using the each of at least two imaging system further includes the at least two imaging systems using at least two of (i) a Digital Breast Tomosynthesis (DBT) system, (ii) a Microwave Nearfield Radar Imaging (NRI) system, (iii) a UltraSound Imaging (USI) system, and a (iv) Thermoacoustic Imaging (TAI) system.

In another aspect, the invention is a cancer detection system, comprising at least two imaging systems, each of which implements an imaging modality different from others of the at least two imaging systems, and each of which provides sampled image data based on its modality. The cancer detection may further include a processor and a memory with computer code instructions stored thereon, the memory is operatively coupled to the processor such that the computer code instructions, when executed by the processor, may cause the system to implement:
(a) for each imaging modality, a modeling unit configured to produce modeled image data for that modality based on a common set of biophysical parameters, (b) a joint non-linear inversion module configured to receive information from each modeling unit and reconstruct a set of joint biophysical properties based on the information from the modeling units, and (c) a scaling unit configured to revise the common set of biophysical parameters based on the set of joint biophysical properties. The cancer detection system may further include a comparator configured to compare the sampled image data from each of the imaging systems to the corresponding modeled image data to determine a difference between the sampled image data and the modeled image data and to determine when the difference is less than a threshold difference, thereby indicating that the sampled image data and the modeled image data has converged. The cancer detection system may further include a classifier configured to classify tissues corresponding to the image data as being normal or in a morphologically atypical state based on the set of joint biophysical properties.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 illustrates an example multimodal cancer detection system constructed according to an embodiment of the invention.

FIG. 2 shows a schematic of the data collection and signal processing of the model-based joint inversion.

FIG. 3 illustrates an example derivation of the upscaling and downscaling techniques according to an embodiment of the invention.

FIG. 4 shows an electrical schematic of an example cancer detection system according to the described embodiments

FIG. 5 summarizes the mathematical formulation of the detection problem according to an embodiment of the invention.

FIG. 6 shows an example embodiment of a cancer detection system constructed according to an embodiment of the invention.

FIGS. 7-12 illustrate numerical results of a processing example according to the described embodiments.

FIGS. 13-18 illustrate numerical results of another processing example according to the described embodiments.

### DETAILED DESCRIPTION OF THE INVENTION

A description of example embodiments of the invention follows.

The teachings of all patents, published applications and references cited herein are incorporated by reference in their entirety.

The described embodiments are directed to a breast cancer detection system that uses a multimodal imaging configuration. The described embodiments may utilize a fusion of two or more imaging modes, including for example (i) Digital Breast Tomosynthesis (DBT), (ii) Microwave Nearfield Radar Imaging (NRI), (iii) Ultrasound Imaging (USI) and Thermoacoustic Imaging (TAI). The described embodiments may evaluate the captured multimodal scan data jointly rather than independently. The described embodiments may further utilize co-registration of the two or more imaging modes, which ensures that the scans of all modes are captured with respect to the same physical configuration of the breast under study, i.e., while the breast is under clinical compression. The co-registration avoids the necessity of registering the images from different independently-operating sensing modalities, which may lead to misfits in the joint inversion of the biophysical parameters.

In standalone imaging systems, the constitutive properties of the target are reconstructed by inverting the forward model operator. For instance, electromagnetic imaging, like NRI, uses the electric and magnetic fields E(r, w) and H(r, w) to estimate the electromagnetic constitutive properties permittivity, magnetic permeability, and electrical conductivity [\dot{o}(r,ω,σ), \hat{μ}(r,ω), \hat{σ}(r,ω)]=Oₑₘ⁻¹{E(r,ω), H(r,ω)}; acoustic imaging, like USI, uses the acoustic pressure p(r,ω) to estimate the acoustic constitutive properties density, the attenuation factor, and compressibility [\hat{ρ}(r), \hat{Q}(r,ω), \hat{κ}(r,ω,Q)]=O_{ac}⁻¹{p(r,ω)}; and x-ray imaging, like 2D mammography and 3D DBT, uses the intensity I(r,ω) to estimate the absorption coefficient [\hat{μ}(r,ω)]=O_{dbt}⁻¹{/(r,ω)}.

In a fused multimode imaging system, a joint inversion operator is O_{ac/em/dbt}⁻¹{·} is used for the same purpose:

[ô(rω,σ), \hat{μ}(r,ω), \hat{σ}(r,ω),ô(r,ω,σ), \hat{μ}(r,ω), \hat{σ}(r,ω), \hat{μ}ₒ(r,ω)]=O_{ac/em/dbt}{p(r,ω), E(r,ω), H(r,ω), H(r,ω)}

This inversion is now jointly performed; and as a result the combined reconstruction is more informative than any one of the sensor alone (since it provides complementary information), more reliable (since it can exploit redundancy in the multiple images), more timely, more accurate and/or less expensive. Therefore multimode imaging has the potential to enable the detection of tumors with better sensitivity and/or specificity, and to allow a better classification of objects since it has access to more features.

Unfortunately, the latter approach does not consider that there is a single underlying physical property that relates all the constitutive properties. Specifically, the constitutive properties of each sensing modalities are related to the biophysical parameters pn through the nonlinear biophysical models Gₑₘ, Gₐₑ, G_{dbt}. In other words, each pixel in the imaging region is made of a mixture of fibrous-connective or glandular tissue, adipose tissue, and in some cases cancerous tissues that is specified by the biophysical parameters vector Pⁿ (note that this tissue mixture is equivalent to use other equivalent parameters like porosity, fluid saturation, and solid matrix composition). This suggests that a direct inversion over the biophysical parameters should be more robust that an inversion over the constitutive parameters. Unfortunately, the biophysical models are non-linear, and ill-posed, which are difficult to invert. The described embodiments provide a solution to this problem by incorporating a robust mathematical formulation and inversion method to jointly invert the nonlinear forward operator and biophysical models in a unified step, so that an enhanced overall sensitivity and specificity are achieved.

Another useful opportunity provided by fused multimode imaging systems is that the information provided by each sensor can be combined in order to classify a pixel or an image region as healthy or tumor. Radiologists often make this decision based on the pixel intensity features of the image (e.g., a strong signal in a DBT image may be indicative of cancer in a fatty breast), morphologic features (e.g., specular morphology in a DBT image may be indicative of cancer in a dense breast), and functional features (e.g., a strong signal is achieved in an MM or PET device after injecting a contrast agent). Notwithstanding, radiologists may not use all the information contained in the reconstructed features when diagnosing a tissue as healthy or tumor. The described embodiments apply novel data analytics and machine learning to improve the sensitivity and specificity of the fused system by incorporating, in addition to intensity features, morphologic features and functional features, patient-specific factors when training the classifier and in the decision (i.e., breast density, family history and genetic testing (e.g., BRACA)) in order to outperform the state of the art.

The multi-modal measurements produced by the multimodal sensing system of the described embodiment is formulated through the nonlinear relationship y=f(x), where x is a vector that is related to the constitutive parameters of the sensing modality, this is ò(r,ω,σ), μ(r,ω), σ(r,ω), ò(r,ω,σ), μ(r,ω), σ(r,ω), μₐ(r,ω); and f(·) is also a nonlinear function of the constitutive parameters that describes the measurement process. The process recovers the vector x from the set of measurements y. Without any prior knowledge about the object of interest, the unknown vector x can take any value permitted by the laws of physics; and, in this case, it is difficult to accurately reconstruct x due to the ill-posed and nonlinear nature of the problem. However, if one introduces additional a priori known information to the problem (i.e., the object is constructed from a mixture of R different tissues, which is determined by the mixture values z₁, z₂, z₃ contained in the vector Pⁿ), one can recover the same problem in a lower dimensional space z₁, z₂, z₃ by considering that the constitutive properties are related to the mixture through the following mapping x=h(z₁, z₂, z₃)—note that h{·}=[Gₑₘ{·}, Gₐₑ{·}, G_{dbt}, {·}]. Since the problem is now resolved in the lower dimensional space, the ill-posedness of the problem is reduced and the reconstruction becomes more stable.

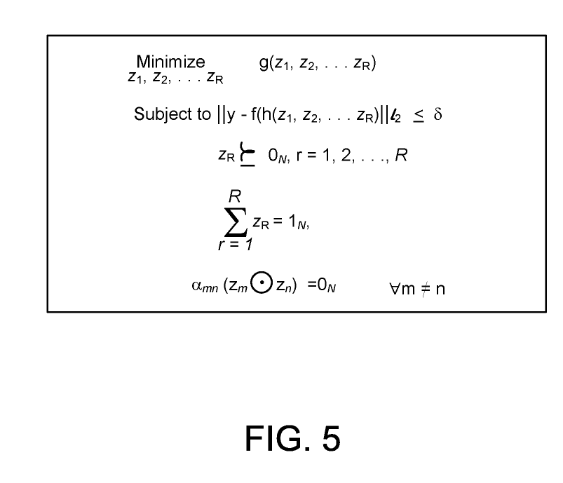

FIG. 5 summarizes the mathematical formulation of this problem, where 0_{N} and 1_{N} are column vectors containing N zeros and ones, respectively, and ⊙ represents a Hadamar (element-wise) product, δ represents an estimate of the error in the measurement vector. The positivity and linear equality constraints ensure that the solution vectors represent valid mixture proportions (non-negative and sum to one), and the nonlinear equality constraint ensures that certain mixtures cannot coexist, when applicable. For example, if a given mixture can only have a component of zₘ or a component of zₙ, but not both simultaneously, then setting Εₘₙ≠0 in the optimization program enforces this constraint. If a mixture of zₘ and zₙ is allowed, then setting αₘₙ=0 permits that possibility.

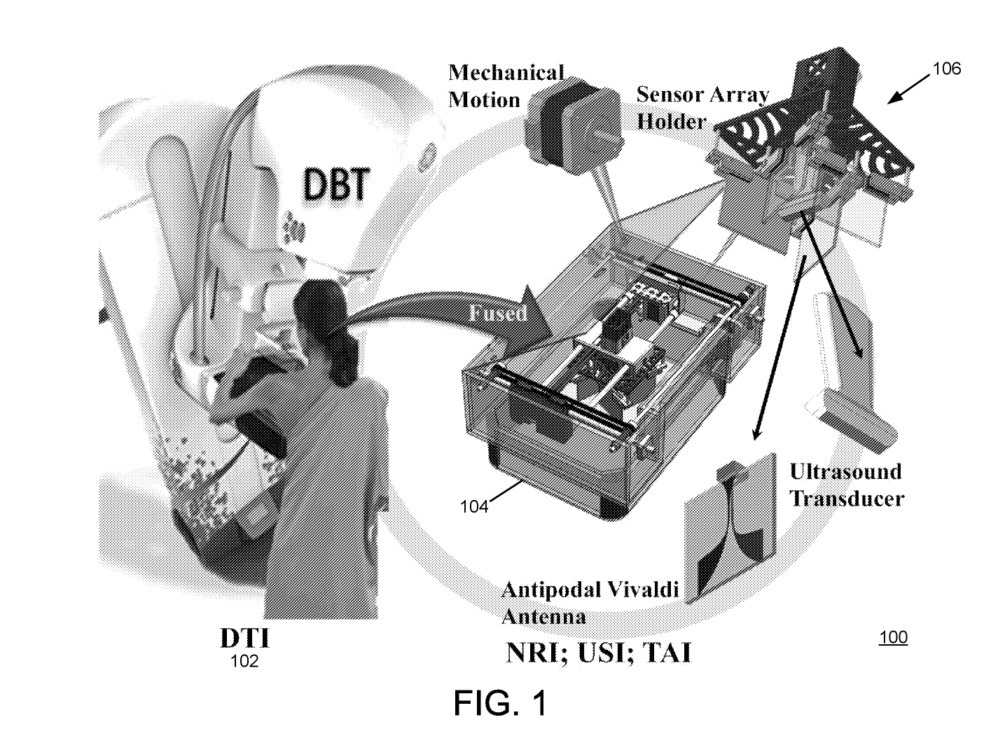

FIG. 1 illustrates a multimodal cancer detection system 100 according to one embodiment. FIG. 1 shows a DBT imager (DTI) 102 coupled with a mechatronic system 104. The mechatronic system 104 includes a sensor array holder 106 that hosts, for example, an NRI, a USI, and a TAI (or other imaging modalities instead of or in addition to these). The mechatronic system 104 may cause the different modalities to mechanically rotate into position (or otherwise be repositioned) for imaging a breast under clinical compression.

The use of a mechatronic system 104, that mechanically translates the NRI/USI/TAI probes (or a subset of them), enables the collection of a large number of measurements, thus reducing the ill-posedness of the collected data and enabling noise reduction by, in some embodiments, averaging consecutive measurements.

The a-priori information used to define a near-to-optimal first guess of the true biological parameters (porosity, fluids saturation, and solid matrix composition) may be obtained, in some embodiments, by inverting the biophysical model of the reconstructed x-ray absorption value at every pixel using a single-modality DBT imaging method. In other embodiments, the first guess of the true biological parameters may be obtained by an imaging modality other than the DBT system (e.g., NRI, USI or TAI).

The fusion of multiple modalities allows the co-registered classification of the biological tissues in terms of nine parameters: (i) electrical permittivity, (ii) permeability (iii) conductivity, (iv) elastic bulk modulus, (v) density, (vi) attenuation, (vii) thermodynamic heat capacity (viii) volumetric expansion coefficient, and (ix) radiological X-ray absorption. These nine features may be used to classify pixels as cancerous or healthy tissues by using basic machine learning (supervised, unsupervised and/or deep learning) classifiers. Additional spatial features may be added to the machine learning classifier. This knowledge may facilitate a breast tissue properties database, which may be used for better refining the biophysical models used during the inversion. A reconstructed vector of unmixed tissues may be used by a machine learning procedure (e.g., simple Quadratic Discriminant Analysis) in order to classify tissues under test as healthy or cancerous.

The joint inversion of the biophysical parameters reduces the dimensionality of the problem, leading to a more suitable inversion when compared with a joint inversion of the constitutive parameters.

The NRI/UST/TAT or the NRI/UST modalities avoid the use of ionizing radiation like the DBT imager, which leads to an imaging technology capable of generating high resolution images (sub-milliliter) showing high contrast between fibrous and cancerous tissues, while keeping the data collection time short (e.g., under 20 seconds).

The multimodal cancer detection system of the described embodiments may operate in a two-step fashion. In the first step, the breast is placed under clinical compression, and DBT measurements are recorded using low-dosage X-rays according to the procedures of the DBT imaging system. In the second step, the mechatronics system 104, which includes the NM, USI and TAT probes immersed in a bolus fluid, is mechanically scanned with respect to the same breast as that breast remains under the same clinical compression observed for the DBT measurements.

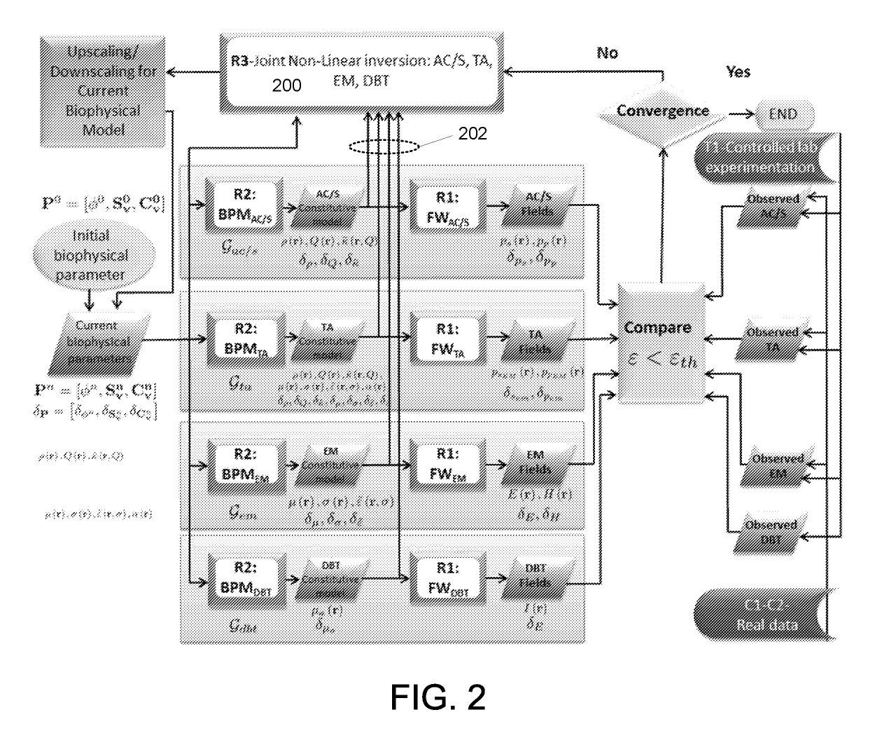

FIG. 2 shows a schematic of the data collection and signal processing method of the model-based joint inversion. The model-based joint inversion 200 starts by defining a near-to-optimal first guess of the biophysical parameters P⁰=[φ⁰, Sᵥ⁰, Cᵥ⁰]. It is useful to note that this model can be obtained, for example, by [symbol] dbt which relates the x-ray absorption μₐ(r) and the biophysical model Pⁿ=[(φⁿ, Sᵥⁿ, Cᵥⁿ], after the DBT imaging method has been used to derive μₐ(r)from the measured DBT projection image I(r). Once the data is collected, a joint 3D reconstruction of the biophysical parameters of the breast is performed. Pⁿ=[φⁿ, Sᵥⁿ, Cᵥⁿ] is the vector containing the porosity, fluid saturation and solid matrix composition parameters at the n-th iteration, which are revised based on the joint biophysical properties 202 from the models. Storage of the data may be done in ways known in the art and may include compression/decompression and/or encryption/decryption techniques.

A set of biophysical models (g_{ac/s}, gₜₐ, g_{cm}, g_{dbt)} are used to relate the biophysical parameters and the traditional constitutive parameters of the different sensing modalities: 1) g_{ac/s} is a function that relates the acoustic parameters ρ(s), Q(r), \hat{κ}(r,Q) (density, attenuation, and bulk modulus) with the biophysical parameters; 2) gₜₐ is a function that relates the thermoacoustic dependent parameters, μ(r), σ(r), \hat{ε}(rσ), α((r), ρ(r), Q(r), \hat{κ}(r,Q) (electric permeability, conductivity, dielectric constant, ratio between heat capacity and compressibility, density, attenuation and bulk modulus) with the biophysical parameters; 3) [symbol] em is a function that relates the electromagnetic parameters μ(r), σ(r), \hat{ε}(r,σ) (permeability, conductivity, and dielectric constant) with the biophysical parameters; and 4) as described above, [symbol] dbt tht is a function that relates the X-ray parameters μₐ(r) and the biophysical parameters.

Once the constitutive parameters are known, a set of forward models (FW_{ACS}, FW_{TA}, FW_{EM}, FW_{DBT}) (acoustics, thermoacoustics, electromagnetic and x-ray DBT) are used to synthetically predict the measured data: a) pressure pₛ(r) of the pₚ and s-waves for the acoustic model; b) electromagnetically induced pressure pₛ_{_{EM}}(r), p_{PEM}(r) of the p- and s-waves for the thermoacoustic model; c) electromagnetic fields E(r), H(r), electric and magnetic, for the microwave sensor and thermoacoustic sensor; d) X-ray intensity I(r) for the DBT sensor. The forward models may include full-wave models, as well as simplified high frequency models based on, for example, rays, currents or Eikonal equations.

If the synthetic data is similar to the measured data (this condition is given by a quadratic data misfit norm as well as a norm-1, norm-2 and/or norm-1,2 regularization term), then the method, which may be iterative, is stopped and Pⁿ=[φⁿ, Sᵥⁿ, Cᵥⁿ] is used to compute all the constitutive parameters for the different technologies. If this condition is not satisfied, a non-linear inversion method (which may be based for instance on Born approximations, iterative born approximations, contrast source methods, Rytov methods, Eikonal inversions using norm-1, norm-2 and/or norm-1,2 regularization terms) can be used to derive the next iteration biophysical parameters Pⁿ=[(φⁿ, Sᵥⁿ, Cᵥⁿ]. Since the inversion may be done at different scales for the different methods, an upscaling/downscaling technique is used to reach an unified-scale biophysical parameters. Multiple iterations of the above processing may be performed until convergence is achieved.

The final biophysical parameters and the nine constitutive parameters (electrical permittivity, permeability conductivity, elastic bulk modulus, density, attenuation, thermodynamic heat capacity volumetric expansion coefficient, and radiological X-ray absorption) may be used (e.g., by supervised, unsupervised and deep learning techniques) in order to classify pixels into the different types of tissues (classes) inside of the breast, which may include (among others) of the following tissues: fatty tissue, fibrous tissue, cancerous tissues, and calcifications.

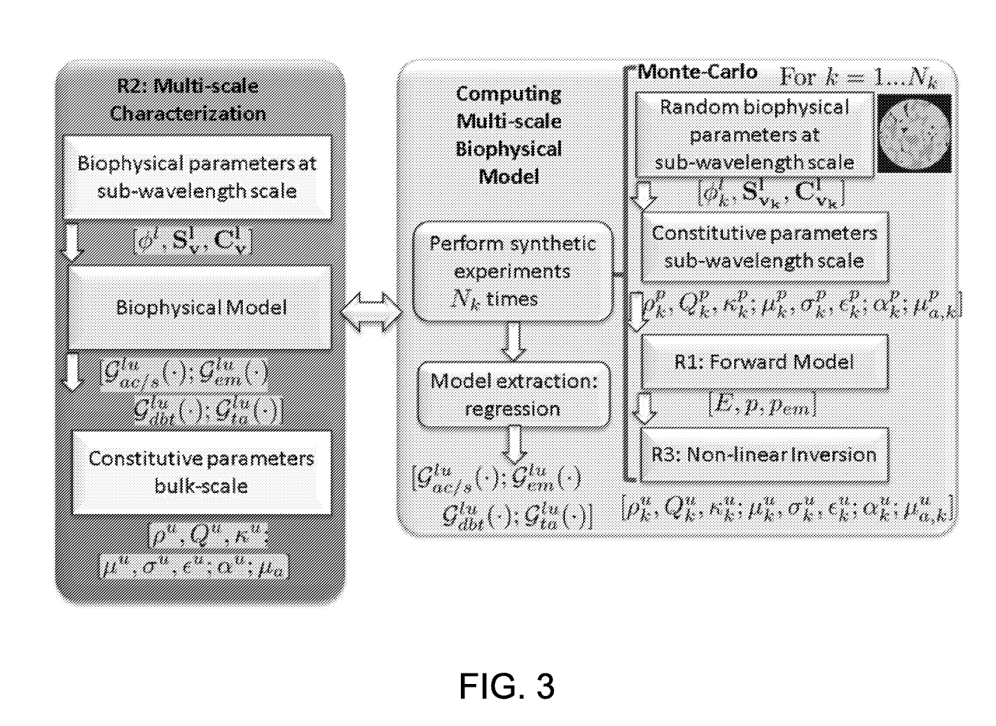

In some embodiments, the biophysical models ( [symbol] _{ac/s} [symbol] ₜₐ, [symbol] ₑₘ [symbol] _{dbt)} and the upscaling and downscaling techniques may be derived from Monte-Carlo simulations, as shown and described in FIG. 3.

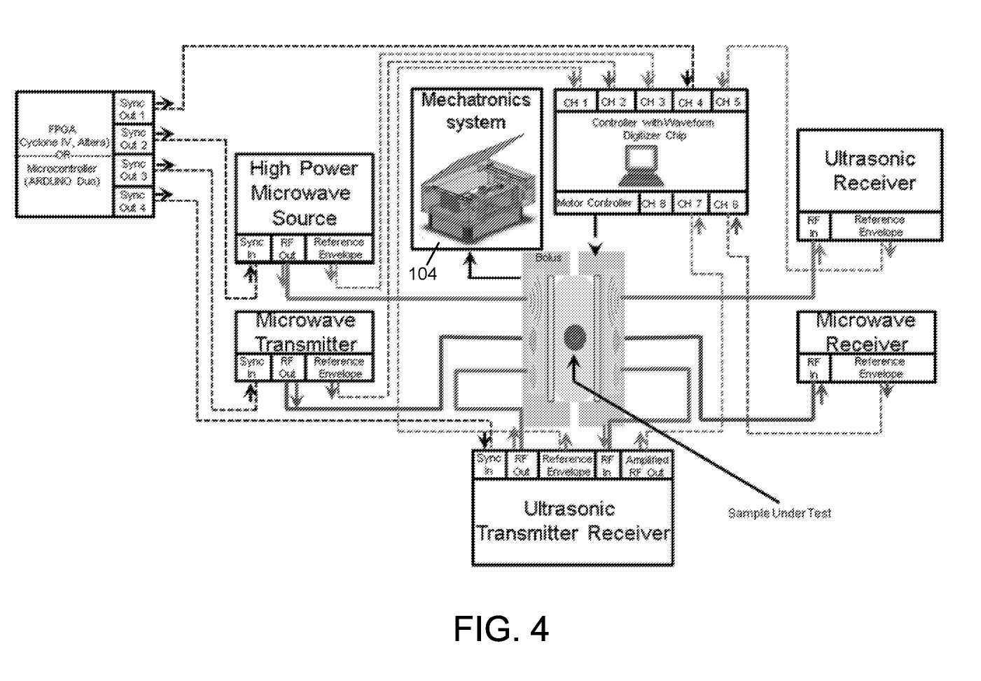

An electrical schematic of an example cancer detection system according to the described embodiments is shown in FIG. 4. Some embodiments may utilize alternative imaging modalities, such as electrical impedance tomography, instead of or in addition to the example modalities shown in FIG. 4 and described herein.

FIG. 5, described elsewhere herein, provides a summary of the multimodal sensing problem.

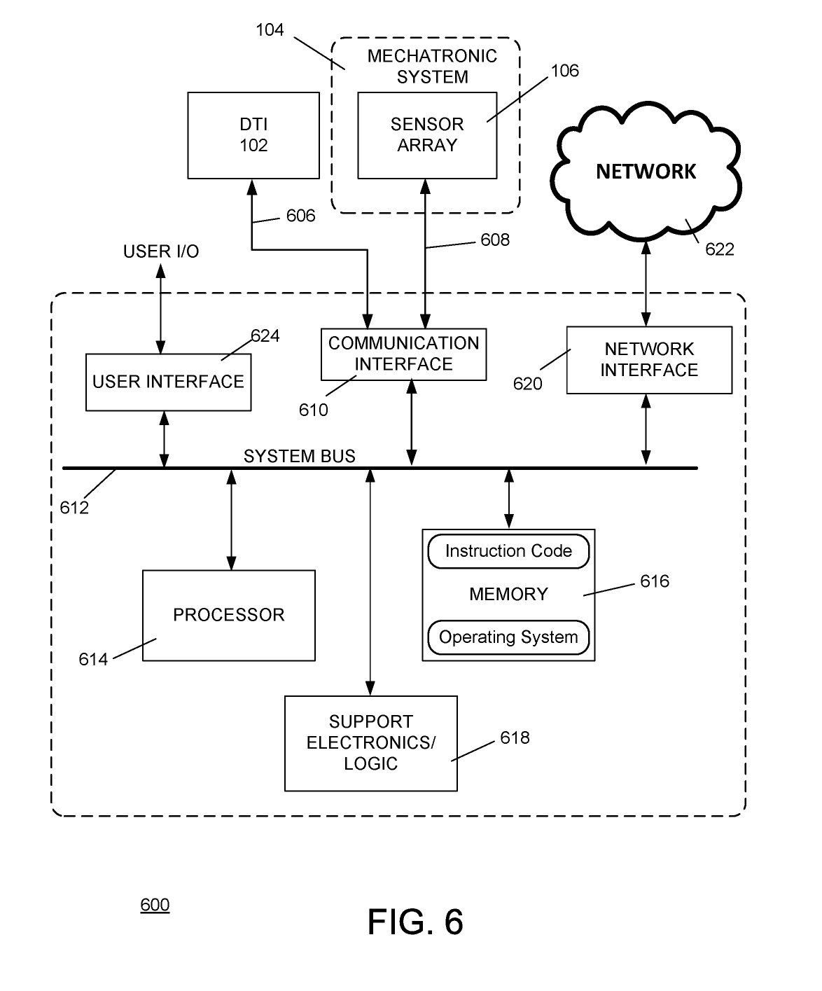

FIG. 6 illustrates an example embodiment of a cancer detection system 600 as described herein. The example system 600 includes a Digital Breast Tomosynthesis Imaging (DTI) system 102, and a mechatronic system 104 that hosts a sensor array 106. The DTI 102 provides image data 606 to a communications interface 610. The sensor array 106 likewise sends image data 608 to the communications interface 610.

The communications interface 610 buffers and formats the image data 606, 608 into a form suitable for transfer to a system bus 612. A processor 614 coordinates with the communications interface 610 to accept the image data and store the information 608 into a memory 616. The system may also include support electronics/logic 618, a network interface 620 for communicating with an external network 622, and a user interface 624 for communicating user information between a system user and the system bus.

The memory 616 also includes instruction code for execution by the processor 614 to perform system operations. The instruction code may include instructions for performing the processing such as image data modeling, joint non-linear inversion, scaling and machine learning, as described herein, and an operating system for coordinating and managing the compressive sensing image processor 626.

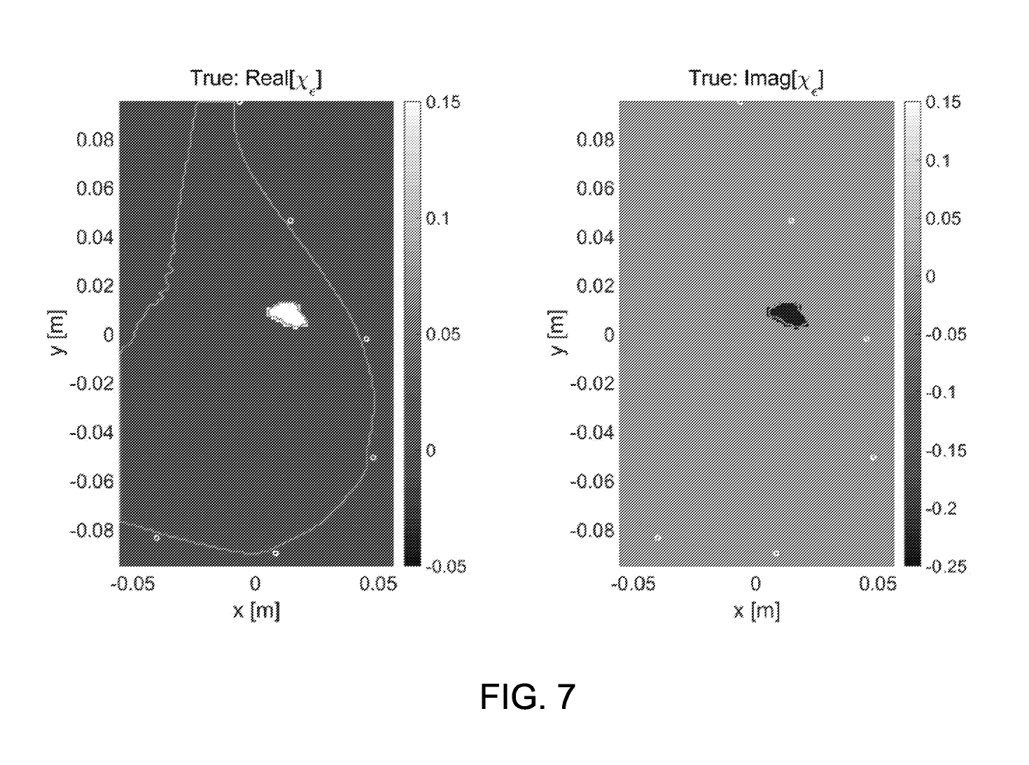

FIGS. 7-12 illustrate numerical results of a processing example according to the described embodiments. In the example, a 2D model of a healthy breast was generated by segmenting a 2D slice from a 3D DBT image. In order to simulate data from a cancerous case, a modeled lesion with frequency-dependent electrical properties was added to the healthy breast. A 2D version of the Finite Differences in the Frequency Domain (FDFD) code was used to generate the synthetic NRI measurements of the healthy breast, the synthetic NRI measurements of the cancerous breast, and the sensing matrix of the healthy breast using the Born approximation. In the simulation, the NRI system used six transmitting and receiving antennas operating in a multi-monostatic configuration. Each antenna was excited with three different frequencies, 500 MHz, 600 MHz, and 700 MHz, for a total of 18 measurements among the antennas.

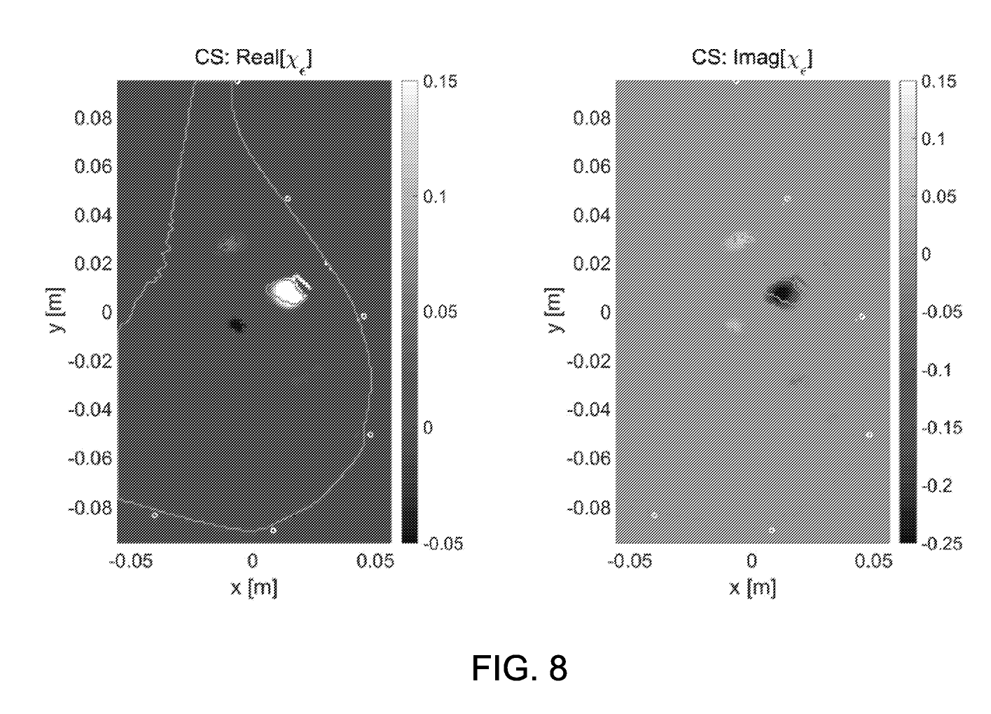

FIG. 7 displays the true contrast variable obtained when the DBT image is segmented perfectly. In this plot, the white dots represent the antenna positions and the curves represent the breast and lesion borders. Since the DBT image was segmented perfectly, the contrast variable is non-zero only at the location of the cancerous lesion. FIG. 8 displays the estimated contrast variable obtained using the perfect DBT segmentation and noiseless measurements. This image, and all subsequent images, were generated by solving the following equation using a value of λ=10⁻⁴.

minimize ½∥Ax−y∥_{R}_{₂}²+λ∥x∥l₁

subject Re(diag(∈_{b})x+∈_{b})≥1

Im(diag(∈_{b})x+∈_{b})≥0

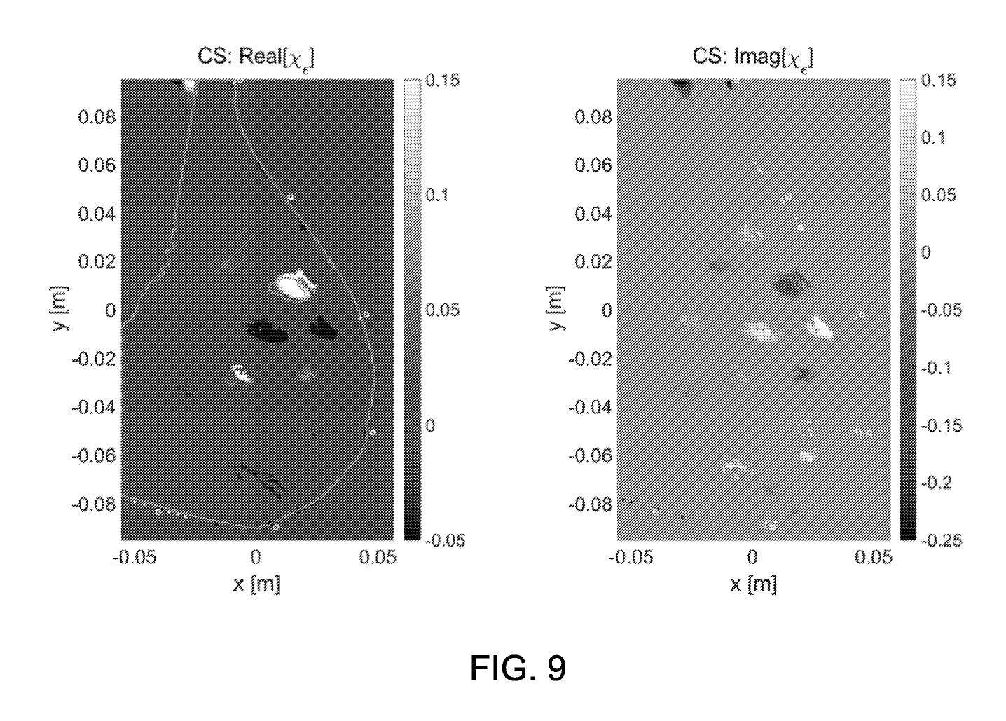

Although there are some artifacts in the image, the algorithm is able to locate the cancerous lesion. FIG. 9 displays the estimated contrast variable obtained using the perfect DBT segmentation and measurements whose SNR=10 dB. Although there are some additional artifacts in the image compared to the noiseless case, the CS-based algorithm is still able to locate the cancerous lesion.

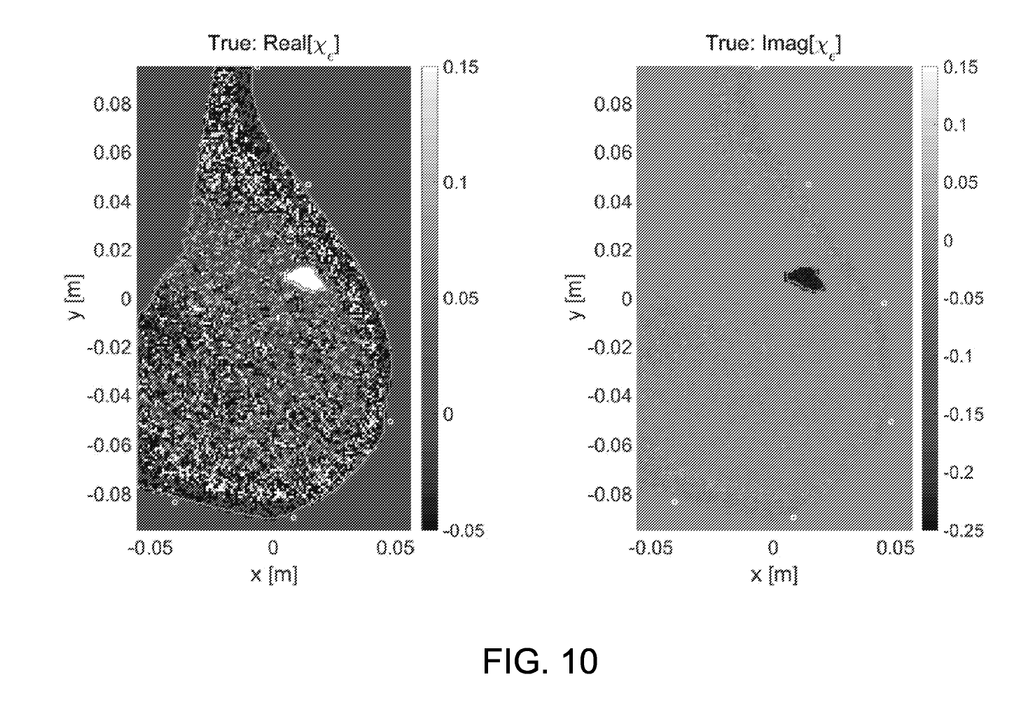

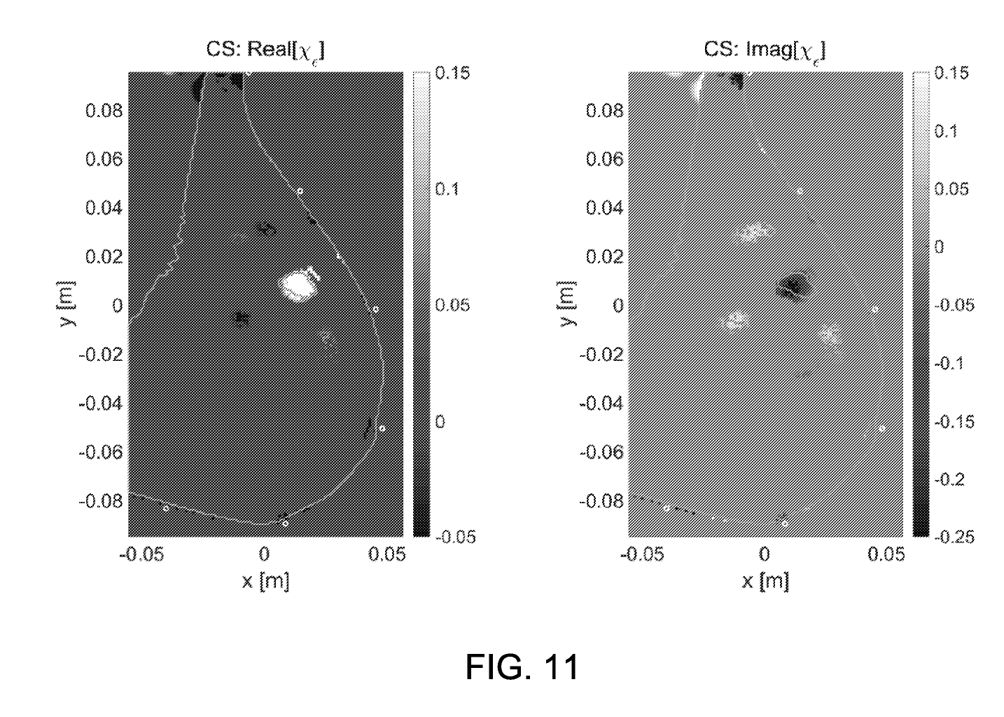

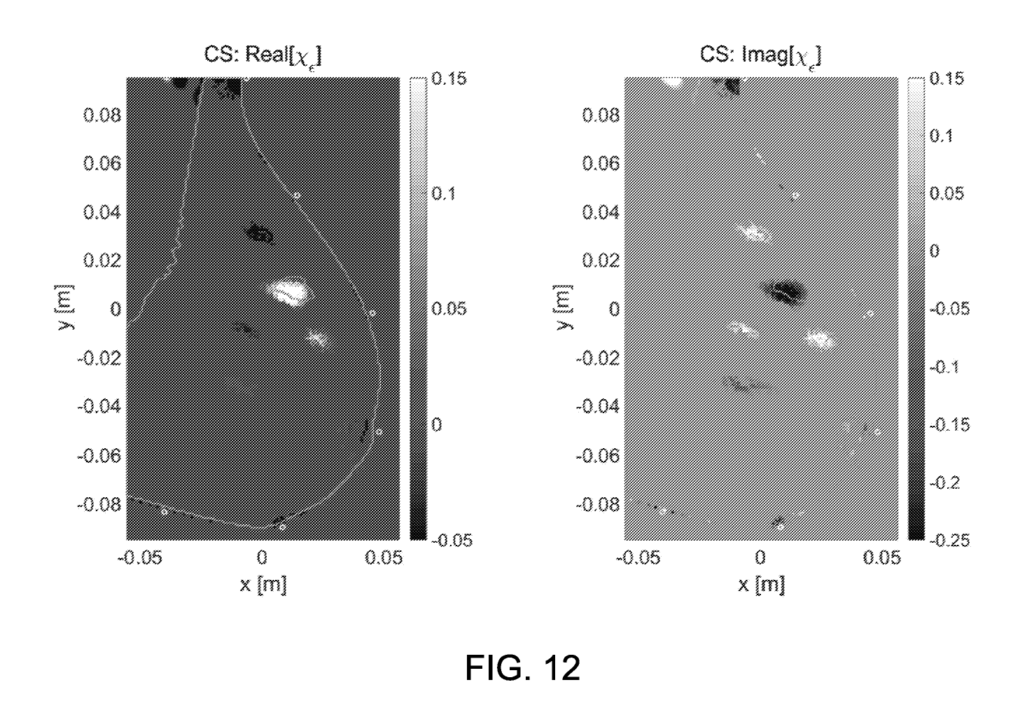

FIG. 10 displays the true contrast variable obtained when the DBT image is segmented with 10% random error. Since the DBT is not segmented correctly, the true contrast variable is non-zero within the healthy tissue. Nevertheless, the true contrast variable is approximately compressible, and so we can still use the equations above to image the breast. This result can be seen in FIG. 11, which displays the estimated contrast variable obtained using the noisy DBT segmentation and noiseless measurements. Finally, FIG. 12 displays the estimated contrast variable obtained using the noisy DBT segmentation and measurements whose SNR=10 dB. Even in the presence of both DBT segmentation and measurement error, the CS-based algorithm is able to localize the cancerous lesion with minimal artifacts.

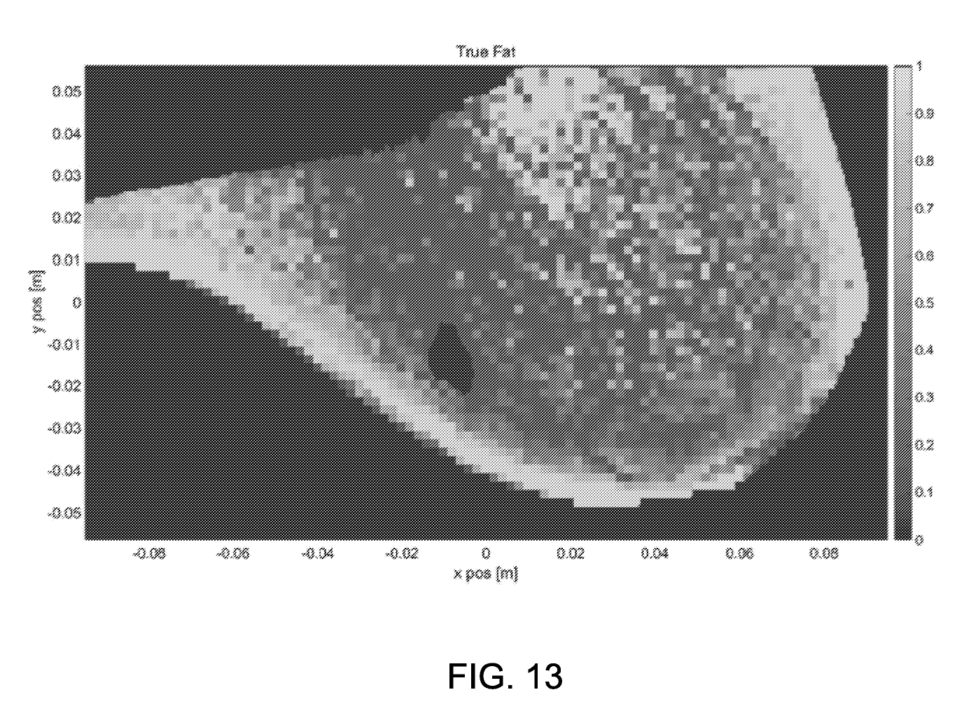

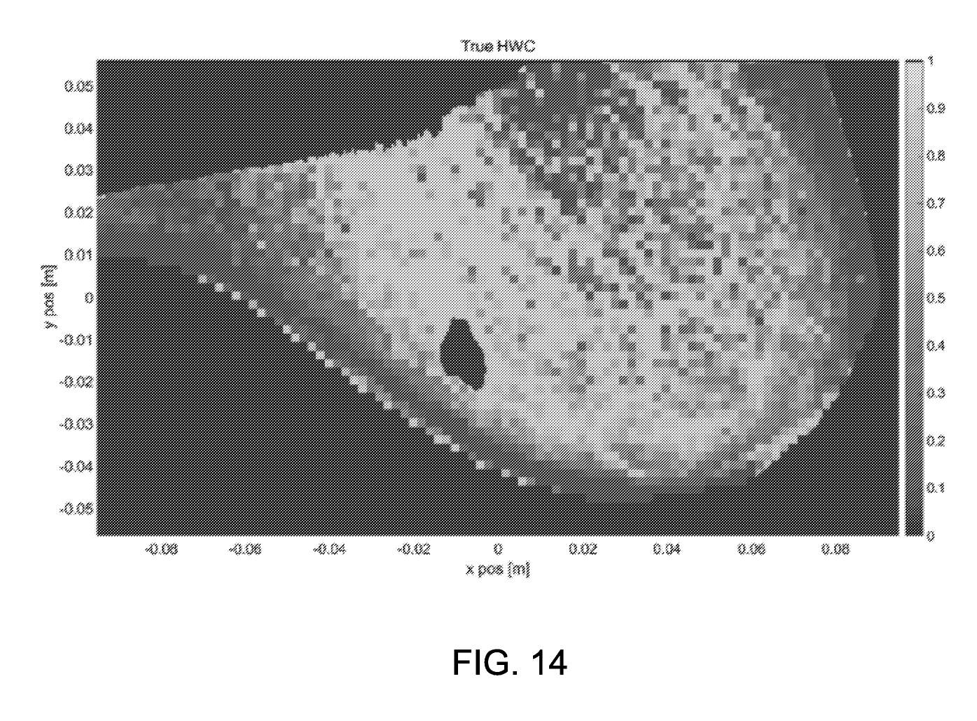

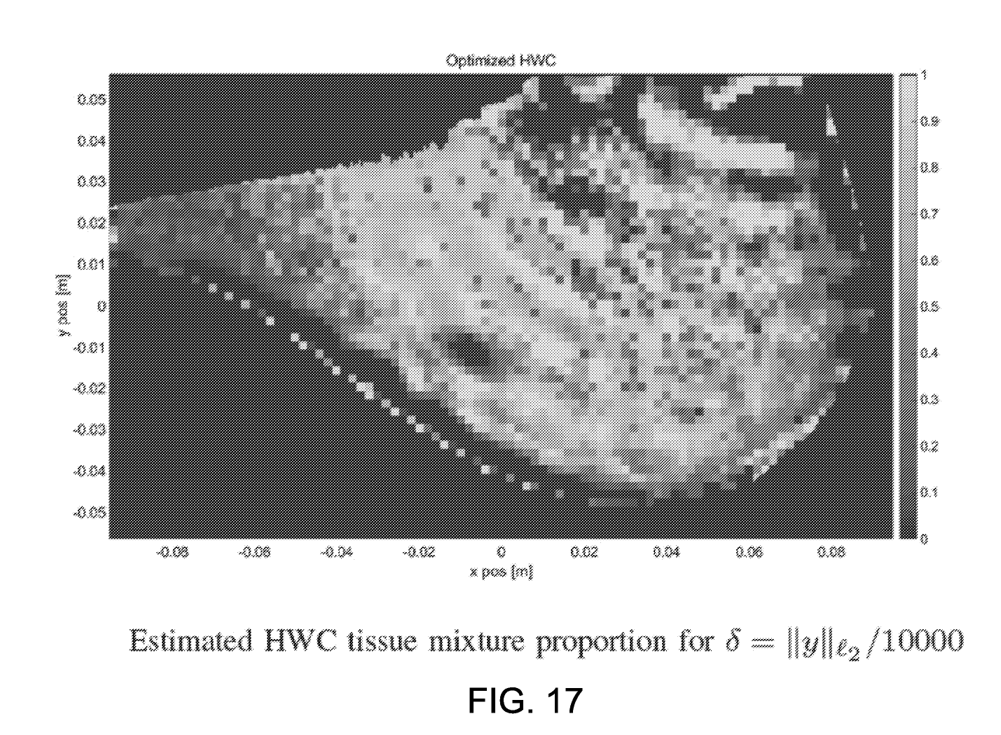

FIGS. 13-18 illustrate numerical results of another processing example according to the described embodiments. A 2D FDFD model was used in order to generate synthetic electric field measurements for two breast geometries, one with a cancerous lesion and one without. The two geometries had the same high water content (HWC) and low water content (LWC) tissue proportions at all locations except for that of the cancerous lesion. The baseline, healthy breast geometry was segmented from a 2D slice of an actual 3D DBT reconstruction. FIGS. 13, 14, and 15 display the true mixture proportions for LWC tissue, HWC tissue, and cancerous tissue, respectively, of the unhealthy breast geometry.

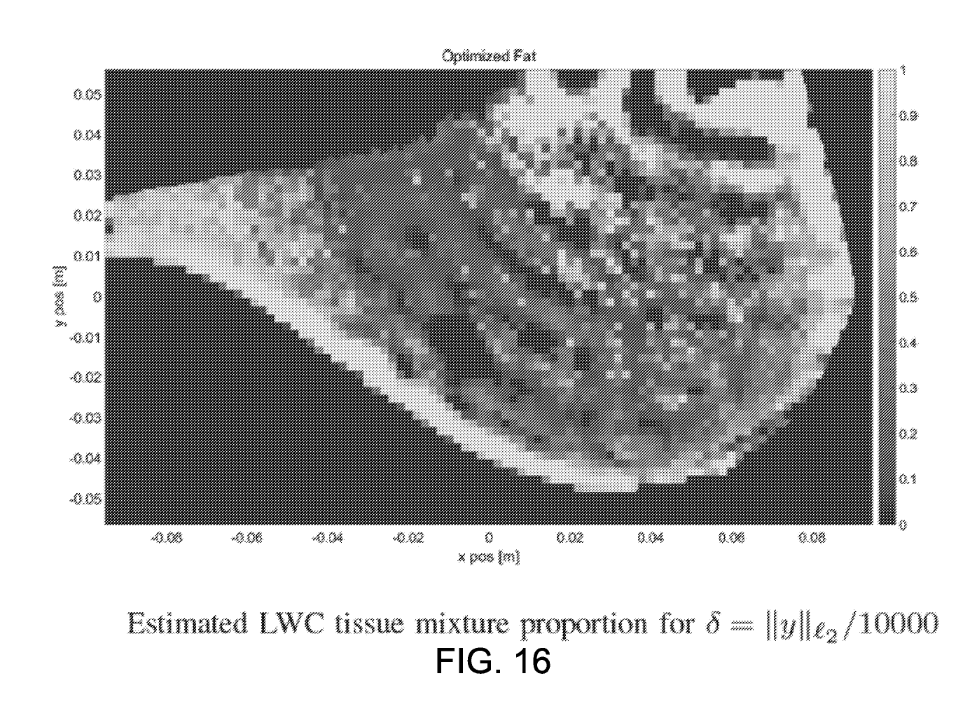

In the numerical simulations, the breast geometries were excited by 17 transmitting and receiving antennas operating in a multistatic configuration. Each transmitting antenna operated at 11 frequencies linearly spaced from 500 MHz to 1500 MHz, for a total of 3179 complex measurements. Note that redundant measurements were used in the optimization routine. The healthy breast geometry simulations were used in order to generate the adjusted measurements ŷ and to compute the Jacobian matrix A required by the optimization procedure. The imaging region was constrained to 9654 positions in the breast, where the grid size of each pixel was 2 mm. In order to consider the problem in the most ideal scenario possible, random noise was not added to the measurements. As a result, the measurements were only corrupted by noise introduced into the problem when it was linearized via the Born Approximation. This noise was estimated to have 12.5% the energy of the adjusted measurement vector, i.e., η≅0.125∥ŷ∥₁₂. In addition, the difference between the measurements of the unhealthy breast, y, and the measurements of the healthy breast, \tilde{y}=f(h(v₁, v₂, v₃)), had approximately 12:69% the energy of the adjusted measurement vector, i.e. ∥y−\tilde{y}∥ₗ_{₂}≈0.1269∥\dot{y}∥ₗ_{₂}. As a result, the parameter in the optimization procedure can be no greater than 0.129∥y∥ₗ_{₂}, otherwise the optimal solution to the problem will be the initial proportions v₁, v₂, v₃. FIGS. 16, 17, and 18 display the estimated mixture proportions for LWC, HWC, and cancerous tissue when δ=∥y∥ₗ_{₂}/10000 is used in the following equation:

$${minimize}_{{z}_{1} , {z}_{2} , {z}_{3}} { \sum }_{r = 1}^{3} {‖ {z}_{r} - {v}_{r} ‖}_{{𝓁}_{1}}$$

$$subject to {‖ \hat{y} - {\sum }_{r = 1}^{3} {A}_{r} {z}_{r} ‖}_{{𝓁}_{2}} \leq δ$$

$${z}_{r} ≽ {0}_{N} , r = 1 , 2 , 3$$

$${\sum }_{r = 1}^{3} {z}_{r} = {1}_{N}$$

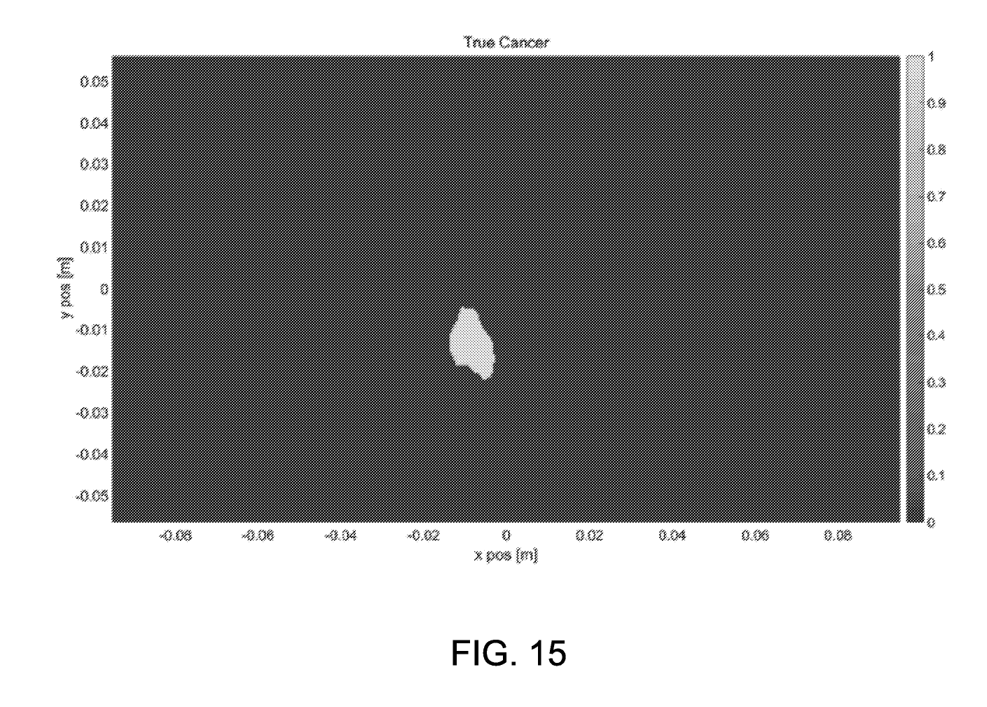

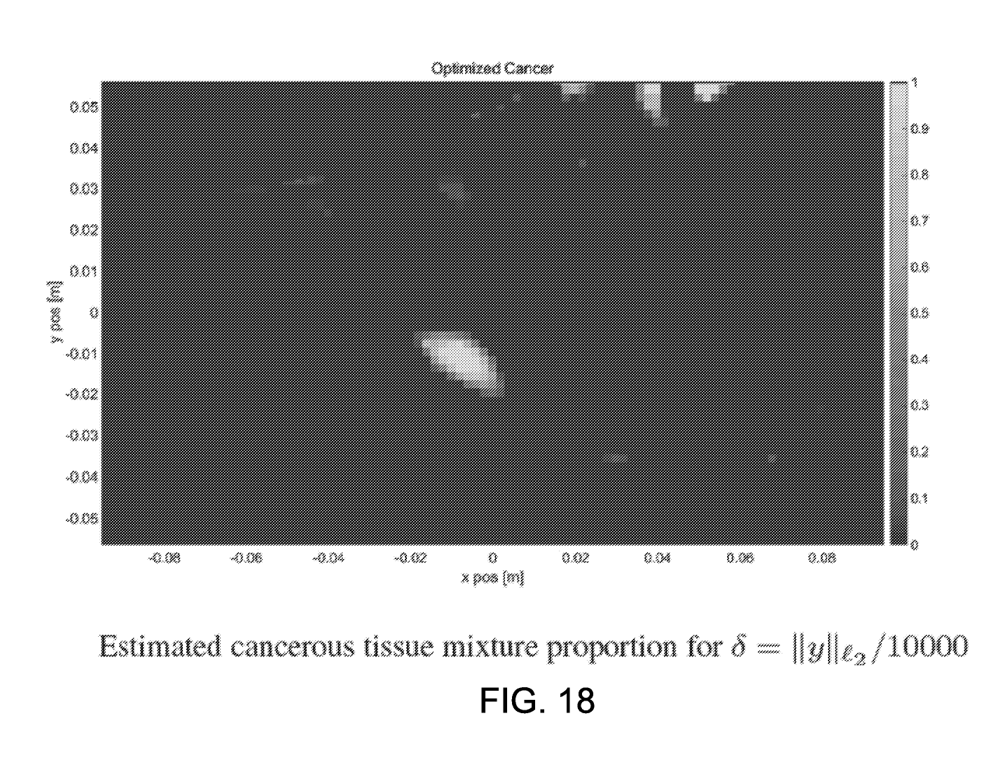

The mixture proportions are not exactly recovered, which is to be expected given that the true solution vector has an error of 0.125∥ŷ∥ₗ_{₂} due to the Born Approximation. Nevertheless, the location of the cancerous lesion within FIG. 18 agrees with the ground truth image of FIG. 15.

It will be apparent that one or more embodiments described herein may be implemented in many different forms of software and hardware. Software code and/or specialized hardware used to implement embodiments described herein is not limiting of the embodiments of the invention described herein. Thus, the operation and behavior of embodiments are described without reference to specific software code and/or specialized hardware it being understood that one would be able to design software and/or hardware to implement the embodiments based on the description herein.

Further, certain embodiments of the example embodiments described herein may be implemented as logic that performs one or more functions. This logic may be hardware-based, software-based, or a combination of hardware-based and software-based. Some or all of the logic may be stored on one or more tangible, non-transitory, computer-readable storage media and may include computer-executable instructions that may be executed by a controller or processor. The computer-executable instructions may include instructions that implement one or more embodiments of the invention. The tangible, non-transitory, computer-readable storage media may be volatile or non-volatile and may include, for example, flash memories, dynamic memories, removable disks, and non-removable disks.

While this invention has been particularly shown and described with references to example embodiments thereof, it will be understood by those skilled in the art that various changes in form and details may be made therein without departing from the scope of the invention encompassed by the appended claims.

What is claimed is:

1. A cancer detection system, comprising:
at least two imaging systems, each of which implements an imaging modality different from others of the at least two imaging systems, and each of which provides sampled image data based on its modality;
a processor; and
a memory with computer code instructions stored thereon, the memory operatively coupled to the processor such that the computer code instructions, when executed by the processor, cause the system to implement:
for each imaging modality, a modeling unit configured to produce modeled image data for that modality based on a common set of biophysical parameters;
a joint non-linear inversion module configured to receive information from each modeling unit and reconstruct a set of j oint biophysical properties based on the information from the modeling units; and
a scaling unit configured to revise the common set of biophysical parameters based on the set of joint biophysical properties;
a comparator configured to compare the sampled image data from each of the imaging systems to the corresponding modeled image data to determine a difference between the sampled image data and the modeled image data and to determine when the difference is less than a threshold difference, thereby indicating that the sampled image data and the modeled image data has converged; and
a classifier configured to classify tissues corresponding to the image data as healthy or cancerous, based on the set of joint biophysical properties corresponding to the modeled image data upon convergence.

2. The cancer detection system of claim 1, wherein the at least two imaging systems includes a Digital Breast Tomosynthesis (DBT) system and a Microwave Nearfield Radar Imaging (NRI) system.

3. The cancer detection system of claim 1, wherein the at least two imaging systems includes two or more of (i) a Digital Breast Tomosynthesis (DBT) system, (ii) a Microwave Nearfield Radar Imaging (NRI) system, (iii) a UltraSound Imaging (USI) system, and a (iv) Thermoacoustic Imaging (TAI) system.

4. The cancer detection system of claim 1, wherein the information from each modeling unit includes biological tissue parameters.

5. The cancer detection system of claim 4, wherein the biological tissue parameters include one or more of (i) electrical permittivity, (ii) permeability (iii) conductivity, (iv) elastic bulk modulus, (v) density, (vi) attenuation, (vii) thermodynamic heat capacity (viii) volumetric expansion coefficient, and (ix) radiological X-ray absorption.

6. The cancer detection system of claim 1, wherein each modeling unit includes a biophysical model, a constitutive model, a forward model, and a field simulating model. The cancer detection system of claim 1, wherein the classifier further classifies tissues corresponding to the image data based on an unmixed version of the set of joint biophysical properties.

8. The cancer detection system of claim 1, wherein classifier utilizes a machine learning procedure to classify the tissues corresponding to the image data.

9. The cancer detection system of claim 1, wherein the classifier utilizes a Quadratic Discriminant Analysis procedure to classify the tissues corresponding to the image data.

10. The cancer detection system of claim 1, wherein the at least two imaging systems reside on a mechatronic system that is integrated with a Digital Breast Tomosynthesis (DBT) system, such that all captured image data is co-registered.

11. A method of detecting cancer, comprising:
using each of at least two imaging systems, performing an imaging modality that is different from others of the at least two imaging systems;
providing, from each of the at least two imaging systems, sampled image data that is based on the image system's modality;
using a processor and a memory with computer code instructions stored thereon,
producing modeled image data, for each imaging modality, based on a common set of biophysical parameters;
reconstructing, based on information received from each modeling unit, a set of joint biophysical properties; and
revising the common set of biophysical parameters based on the set of joint biophysical properties;
comparing the sampled image data from each of the imaging systems to the corresponding modeled image data to determine a difference between the sampled image data and the modeled image data and determining when the difference is less than a threshold difference, thereby indicating that the sampled image data and the modeled image data has converged; and
classifying tissues corresponding to the image data as healthy or cancerous, based on the set of joint biophysical properties corresponding to the modeled image data upon convergence.

12. The method of claim 11, further including sequentially activating each of the imaging systems while a test subject remains clinically advantageous position.

13. The method of claim 12, further including mechanically rotating sensors of the two or more imaging systems, in conjunction with the activating, to accomplish co-registration of the two or more imaging systems.

14. The method of claim 11, further including classifying tissues corresponding to the image data using a machine learning procedure.

15. The method of claim 11, further including classifying tissues corresponding to the image data using a Quadratic Discriminant Analysis procedure.

16. The method of claim 11, further including implementing, for each imaging modality, a forward model that simulates fields corresponding to the imaging modality.

17. The method of claim 11, further including repeatedly revising the common set of biophysical parameters until the difference between the sampled image data and the modeled image data is less than a threshold difference.

18. The method of claim 11, wherein the set of joint biophysical properties include one or more of (i) electrical permittivity, (ii) permeability (iii) conductivity, (iv) elastic bulk modulus, (v) density, (vi) attenuation, (vii) thermodynamic heat capacity (viii) volumetric expansion coefficient, and (ix) radiological X-ray absorption.

19. The method of claim 11, wherein processing modeled image data for each modality is accomplished with a biophysical model, a constitutive model, a forward model, and a field simulating model.

20. The method of claim 11, wherein performing the imaging modality using the each of at least two imaging system further includes the at least two imaging systems using at least two of (i) a Digital Breast Tomosynthesis (DBT) system, (ii) a Microwave Nearfield Radar Imaging (NRI) system, (iii) a UltraSound Imaging (USI) system, and a (iv) Thermoacoustic Imaging (TAI) system.

21. A system for distinguishing a state of human or animal cells from a normal state, the system comprising:
at least two imaging systems, each of which implements an imaging modality different from others of the at least two imaging systems, and each of which provides sampled image data based on its modality;
a processor; and
a memory with computer code instructions stored thereon, the memory operatively coupled to the processor such that the computer code instructions, when executed by the processor, cause the system to implement:
for each imaging modality, a modeling unit configured to produce modeled image data for that modality based on a common set of biophysical parameters;
a joint non-linear inversion module configured to receive information from each modeling unit and reconstruct a set of j oint biophysical properties based on the information from the modeling units; and
a scaling unit configured to revise the common set of biophysical parameters based on the set of joint biophysical properties;
a comparator configured to compare the sampled image data from each of the imaging systems to the corresponding modeled image data to determine a difference between the sampled image data and the modeled image data and to determine when the difference is less than a threshold difference, thereby indicating that the sampled image data and the modeled image data has converged; and
a classifier configured to classify tissues corresponding to the image data as being normal or in a morphologically atypical state based on the set of joint biophysical properties.
